(19) United States
(12) Patent Application Publication
(10) Pub. No.: US 2022/0370809 A1
(43) Pub. Date: Nov. 24, 2022

(54) NAILHEAD FEEDTHROUGH

(71) Applicant: Medtronic, Inc., Minneapolis, MN (US)

(72) Inventors: Joseph J. Viavattine, Vadnais Heights, MN (US); Brad C. Tischendorf, Minneapolis, MN (US); Hailiang Zhao, Plymouth, MN (US)

(21) Appl. No.: 17/316,921

(22) Filed: May 20, 2021

(51) Int. Cl. A61N 1/375

(52) U.S. Cl. CPC A61N 1/3754; A61N 1/37512

(57) ABSTRACT

A battery comprising a battery cell within a battery housing. The battery further comprises a feedthrough inserted through an opening in a top cover of the battery. The feedthrough including a nailhead. A diameter of the nailhead is substantially the same as a length of the feedthrough. Methods for manufacturing the battery are also described.

### FIELD

The present technology is generally related to batteries for use with implantable medical devices. More specifically, the present technology relates to design of feedthroughs for providing electrical contact to a battery cell through a battery top cover.

### BACKGROUND

As implantable medical device (IMD) technology advances, issues such as IMD battery longevity, IMD size and shape, IMD mass, and patient comfort remain key considerations in the IMD design process. Battery size and capacity, for example, significantly impact the physical configuration of the IMD and the duration of service time within the patient before battery replacement or recharge is required. Batteries can include nailhead feedthroughs to provide for electrical contact to the battery cell. However, long and thin nailheads may break off or bend, causing electrical shorts and other failures.

### SUMMARY

The techniques of this disclosure generally relate to battery apparatuses.

In one aspect, the present disclosure provides a battery having a battery cell within a battery housing. The battery further includes a feedthrough inserted through an opening in a top cover of the battery. The feedthrough can include, at least at one end of the feedthrough, at least one of a shoulder feature and a nailhead. The nailhead can have substantially the same length as a length of the feedthrough. The feedthrough can further comprise a pin having a pin diameter smaller than the diameter of the nailhead.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a diagram of an example therapy system including an implantable cardiac device (ICD).

FIG. 2 is a block diagram of an ICD that includes a battery in accordance with embodiments.

FIG. 3A is an exploded view of battery components in accordance with embodiments.

FIG. 3B is a view of an alternative battery housing in accordance with embodiments.

FIG. 4A is an exploded view of a top cover and feedthrough pin in accordance with embodiments.

FIG. 4B is a perspective view of a top cover and nailhead feedthrough in accordance with embodiments.

FIG. 5A is a side view of a nailhead feedthrough for illustrating dimensions of a nailhead feedthrough in accordance with embodiments.

FIG. 5B is a top view of a nailhead feedthrough for illustrating dimensions of a nailhead feedthrough in accordance with embodiments.

FIG. 6A is a side view of a nailhead feedthrough pin according to a first example embodiment.

FIG. 6B is a side view of a nailhead feedthrough pin according to a second example embodiment.

FIG. 6C is a side view of a nailhead feedthrough pin according to a third example embodiment.

FIG. 6D is a side view of a nailhead feedthrough pin according to a third example embodiment.

FIG. 7A illustrates a first assembly option in accordance with embodiments.

FIG. 7B illustrates a second assembly option in accordance with embodiments.

FIG. 7C illustrates a third assembly option in accordance with embodiments.

FIG. 7D illustrates a fourth assembly option in accordance with embodiments.

FIG. 7E illustrates a fifth assembly option in accordance with embodiments.

FIG. 7F illustrates a sixth assembly option in accordance with embodiments.

FIG. 7G illustrates a seventh assembly option in accordance with embodiments.

FIG. 8 is a flow diagram of a method for manufacturing a battery in accordance with embodiments.

### DETAILED DESCRIPTION

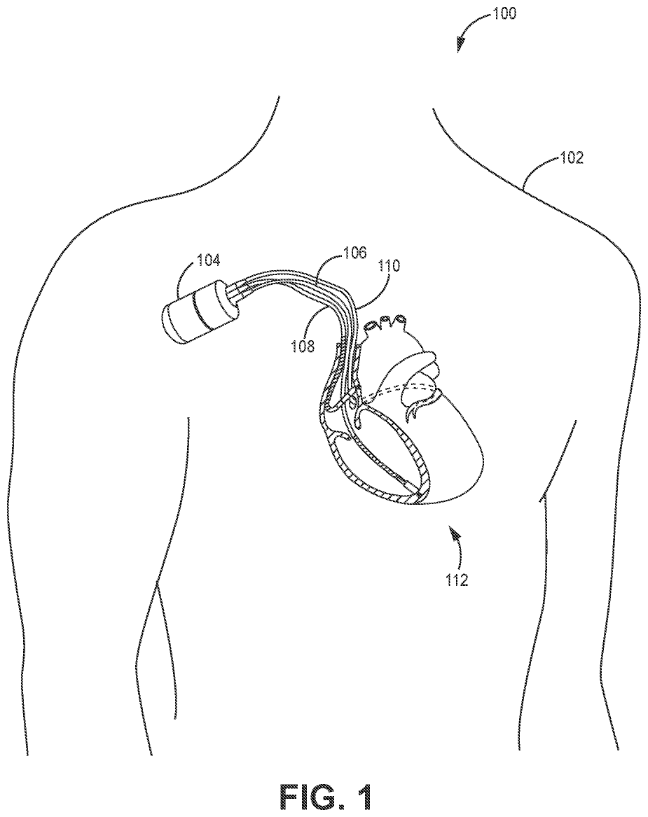

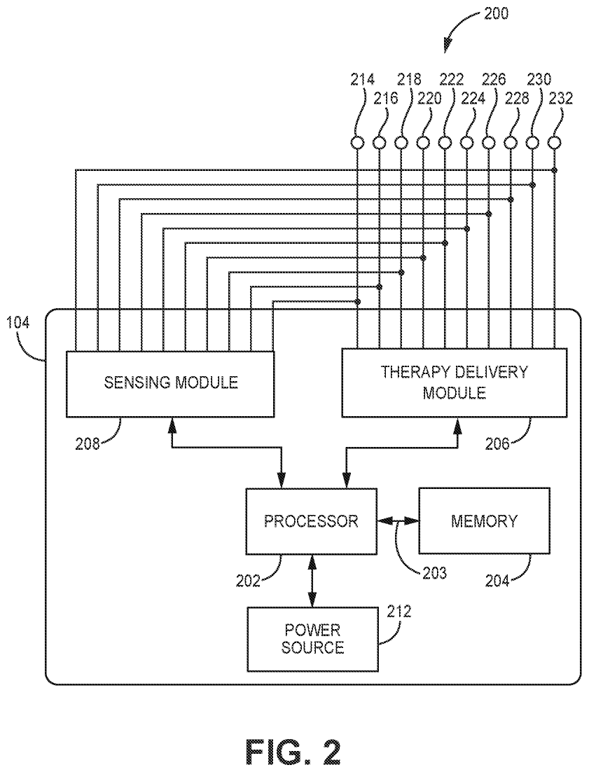

The batteries described herein may be used in any suitable device, such as an implantable medical device. Examples of suitable implantable medical devices include implantable devices that provide therapy to, or sense signals from, a heart of a patient; implantable devices that provide therapy to, or sense signals from, a portion of a central or peripheral nervous system of a patient, implantable devices that deliver therapeutic fluids to a patient, and the like. More specific examples of implantable medical devices that may employ batteries as described herein include implantable pacemakers, cardioverters, defibrillators, deep brain stimulators, spinal cord stimulators, and drug pumps. For purposes of context, an implantable cardiac device (ICD) is discussed regarding FIGS. 1-2 below.

FIG. 1 is a conceptual diagram illustrating an example system 100 that provides therapy to patient 102. Therapy system 100 includes ICD 104, which is connected to leads 106, 108 and 110. ICD 104 may be, for example, a device that provides cardiac rhythm management therapy to heart 112, and may include, for example, an implantable pacemaker, cardioverter, and/or defibrillator that provide therapy to heart 112 of patient 102 via electrodes coupled to one or more of leads 106, 108 and 110. Leads 106, 108, 110 extend into the heart 112 of patient 102 to sense electrical activity of heart 112 and/or deliver electrical stimulation to heart 112.

FIG. 2 is a block diagram of an ICD 104 that includes a power source 212 comprising a battery in accordance with embodiments. The ICD 200 includes a processor 202, memory 204, stimulation generator 206, sensing module 208, and power source 212. The processor 202 may communicate with memory 204 over an interconnect 203 (e.g., a bus). The interconnect 203 may include any number of technologies, including industry standard architecture (ISA), extended ISA (EISA), peripheral component interconnect (PCI), peripheral component interconnect extended (PCIx), PCI express (PCIe), or any number of other technologies. The interconnect 203 may be a proprietary bus.

Stimulation generator 206 is electrically coupled to electrodes 214, 216, 218, 220, 222, 224, 226, 228, 230, 232 e.g., via conductors of the respective lead 106, 108, 110, or, in the case of housing electrode 230, via an electrical conductor disposed within housing of ICD 104. Stimulation generator 206 is configured to generate and deliver electrical stimulation therapy to heart 112 to manage a rhythm of heart 112. Electrodes 214, 216, 218, 220, 222, 224, 226, 228, 230, 232 can include ring electrodes or helical electrodes, for example, although embodiments are not limited thereto. Sensing module 208 monitors signals from at least one of electrodes 214, 216, 218, 220, 222, 224, 226, 228, 230, 232 to monitor electrical activity of heart 112, e.g., via an EGM signal.

The various components of ICD 104 are coupled to power source 212, which may include a rechargeable or non-rechargeable battery. For example, the processor 202 may be coupled to receive power from the power source 212. A non-rechargeable battery may be selected to last for several years, while a rechargeable battery may be inductively charged from an external device, e.g., on a daily or weekly basis. Examples of a rechargeable battery include, but are not limited to, a lithium-ion battery, a lithium/silver vanadium oxide battery, a lithium polymer battery, or a supercapacitor.

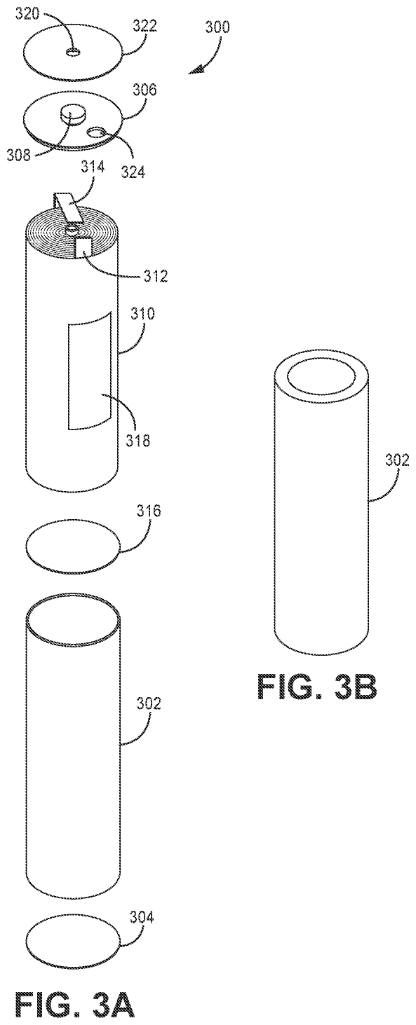

FIG. 3A is an exploded view of battery 300 components in accordance with embodiments. Power source 212 may include a battery housing 302. The battery housing 302 can comprise metallic alloys and provide the ground or negative terminal of a battery 300. Alternatively, the battery housing 302 can be at a positive potential and a feedthrough 308 (described in more detail later herein) can be at a negative potential.

The battery housing 302 can have an open first end and an open second end and the battery housing 302 can be substantially cylindrical having a uniform inner diameter, a uniform outer diameter and uniform wall thickness throughout a length of the battery housing 302. While the battery housing 302 is shown and described as having a generally cylindrical shape, however, the battery housing 302 can have other cross-sectional shapes including, but not limited to rectangular, triangular, square, hexagonal, and octagonal shapes. As referred to herein, the term tubular does not indicate to any particular cross-sectional shape, but only indicates a component including a hollow elongated body.

Alternatively, a battery housing 326 can be formed in a deep draw process in which the one of the first end and second end is closed and the corresponding cover is instead formed as one piece with the battery housing 326, as shown in FIG. 3B. Other components of FIG. 3A can be included within the one-piece battery housing 326, including, for example, battery cell 310 and other components.

The battery housing 302 can have a length greater than its diameter. As examples, the length of the battery housing 302 can be about 1.1 times to about 10 times the diameter of the battery housing 302. As an example, the length of the battery housing 302 can be about 50-70 millimeters and the diameter of the battery housing can be about 15-25 millimeters. In examples, the battery housing 302 can be about 65 millimeters in length and about 19 millimeters in diameter.

The battery housing 302 having an open first end and an open second end can be formed by any suitable process. For example, the battery housing 302 can be formed by extruding or rolling and seam sealing, which removes the need for drying or other processes associated with deep drawing. The battery housing 302 can be formed in a machining process from a solid base stock. The battery housing 302 can be formed from a drawn tubing. Shrink wrapping or other surface can be provided over the battery housing 302. The shrink wrapping can prevent electrical shorting and provide an insulator for the battery. The shrink wrapping can be heat shrunk to the outer surface of the battery housing 302. In some examples, such a shrink wrapping can be applied around the battery 300 after assembly.

The bottom cover 304 may be coupled to the battery housing 302 in any suitable manner. For example, the bottom cover 304 may be coupled to the battery housing by welding. The battery 300 can include a top cover 306. The top cover 306, the bottom cover 304, and the battery housing 302 may have any suitable thicknesses and can be the same or different. In some examples, walls of a battery housing 302 can be about 0.008 to 0.016 inches (or 0.2 to 0.4 millimeters) thick. In some examples, the top cover 306 can be about 0.5 inches (or 12.7 millimeters) thick. The top cover 306 can include feedthrough 308 to provide electrical contact to the battery cell 310. The top cover 306 can be made thinner if feedthrough 308 is not integrated into the top cover 306. For example, the top cover 306 can be about 0.008 to 0.07 inches (or 0.2 to 1.778 millimeters) thick in absence of a feedthrough. The bottom cover 304 can be about 0.008-0.04 inches (or 0.2 to 1.016 millimeters) thick. In examples, the bottom cover 304 can be thinner than the walls of the battery housing 302. The top cover 306, the bottom cover 304, and the battery housing 302 can all be of same thicknesses as each other in some embodiments. In some embodiments, any of the top cover 306, the bottom cover 304 and the battery housing 302 can be thinner or thicker than any other of the top cover 306, bottom cover 304 and battery housing 302. This allows for independent design of each of the top cover 306, bottom cover 304 and battery housing 302.

Similarly to the battery housing 302, the top cover 306, and bottom cover 304 can comprise metallic alloys and provide the ground or negative terminal of the tubular battery. Alternatively, the battery housing 302 can be at positive potential with the feedthrough 308 being at negative potential. The battery housing 302 can be welded to bottom cover 304 and top cover 306 or otherwise attached to form a substantially-sealed enclosure encasing battery cell 310.

Battery cell 310 is depicted as being arranged in a jelly roll configuration with tabs 312 and 314, although embodiments are not limited to a jelly roll configuration for battery cell 310. In a jelly roll configuration, an insulating sheet (not shown in FIG. 3A) is laid down, then a thin layer (not shown in FIG. 3A) of an anode material is laid down, a separator layer is applied, and a cathode material is layered (not shown in FIG. 3A) on top. The layers are rolled and inserted into battery housing 302. A bottom insulator 316 can prevent the battery cell 310 from touching or contacting the bottom cover 304. In an example, one tab 312 may connect to cathode material, and the other tab 314 may connect to anode material of the battery cell 310. Battery cell 310 may comprise lithium/silver vanadium oxide, for example. Alternatively, battery cell 310 can be rechargeable and anodes and cathodes can comprise other materials besides lithium/silver vanadium oxide. Adhesive tape 318 can be included to hold the outer edge of the jelly roll in place.

Top cover 306 includes feedthrough 308 to provide electrical contact to the battery cell 310 through hole 320. Insulator 322 is applied over the top cover 306. Opening 324 allows access for an electrolyte to be provided to the battery cell 310 before the top cover 306 is welded or otherwise attached to the battery housing 302. In some examples, as mentioned earlier herein with reference to FIG. 3A, shrink wrapping can be applied over the entire battery 300. With increased current capacity of some battery cells 310, the feedthrough 308 will need to carry higher current (e.g., 0.1 to 10 Amperes (A)) without, however, adding excessive resistance.

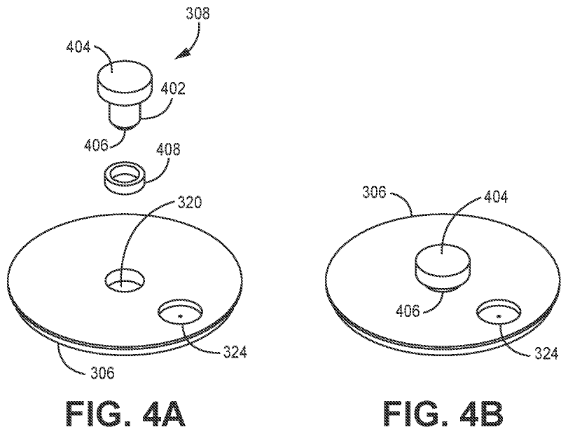

FIG. 4A is an exploded view of a top cover 306 and feedthrough 308 in accordance with embodiments. The feedthrough 308 can carry relatively higher currents without adding resistance due to dimensions of the feedthrough 308 as will be described later herein. The feedthrough 308 can be comprised of titanium, for example grade 23 titanium, stainless steel, or other material that is resistant to corrosion. The top cover 306 can act as a negative terminal or portion of a negative terminal of battery 300. The feedthrough 308 can be at a positive potential and therefore form the positive terminal or portion of the positive terminal of the battery 300. Accordingly, for proper battery 300 operation, the feedthrough 308 should not contact the top cover 306. A glass preform 408 can provide electrical isolation between the top cover 306 and the feedthrough 308.

The feedthrough 308 includes a pin 402 having a nailhead 404 at a first end, and a shoulder feature 406. The shoulder feature 406 can be included at a second end of pin 402 or at the nailhead 404, or both. The shoulder feature 406 and nailhead 404 can improve the ease with which the feedthrough 308 can be manufactured, while preventing failure modes such as the breaking or bending of the feedthrough 308. Breaking and other failure modes can also be prevented by providing pin 402 and nailhead 404 having specific dimensions as described later herein. Generally, the ratio of the pin diameter to pin length can be larger than in other available feedthroughs, which increases stiffness of the feedthrough 308. The shoulder feature 406 can be used to fit feedthrough 308 into an opening or hole, such as could be formed by a jumper mechanism or for interfacing with a plate for ease of welding during manufacturing.

FIG. 4B is a perspective view of a top cover 306 and nailhead 404 of a feedthrough 308 in accordance with embodiments. During manufacturing, the glass preform 408 can be heated and then cooled, forming a hermetic seal over the hole 320 (not shown in FIG. 4B). Subsequent to assembly as partially shown in FIG. 4B, therefore, a hermetic seal and electrical isolation are achieved between the top cover 306 and feedthrough 308. In examples, the hermetic seal formed by glass preform 408 can be such that a leak rate is less than about 5×10⁻⁹ cubic centimeters per second (cc/sec). The pin 402 will be substantially perpendicular to the top cover 306 subsequent to assembly.

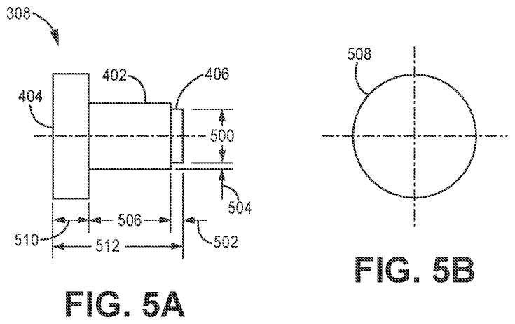

FIG. 5A is a side view of a feedthrough 308 for illustrating dimensions of a feedthrough 308 in accordance with embodiments. Shoulder feature 406 can have a diameter 500 of about 0.0749 to 0.0751 inches. Ideally, the shoulder feature 406 can have a diameter 500 of about 0.075 inches. The shoulder feature 406 can have a length 502 of about 0.009 to 0.011 inches. Ideally, the shoulder feature 406 can have a length 502 of about 0.01 inches. A ratio of shoulder feature 406 diameter 500 to shoulder feature 406 length 502 can be about 7.5:1.

Pin 402 can have a diameter 504 of about 0.0955 to 0975 inches. Ideally, the pin 402 can have a diameter 504 of about 0.0965 inches, or about four to five times the diameter of pins in currently available battery systems. The increased diameter 504 of pin 402 can enable the pin 402 to carry more current without increasing resistance. The pin 402 can have a length 506 of about 0.113 to 0.115 inches. Ideally, the pin 402 can have a length 506 of about 0.114 inches. A ratio of pin 402 diameter 504 to pin 402 length 506 can be about 7.5:1.

Referring also to FIG. 5B, the feedthrough 308 can therefore have a total diameter 508 equal to the diameter 508 of the nailhead 404, or about 0.175 inches. The nailhead 404 can have a length 510 of about 0.049 to 0.051 inches or ideally about 0.050 inches. The nailhead 404 can provide a surface area for welding or other interconnecting process, and the nailhead 404 can provide a mechanism for retaining insulators or other materials over the battery 300. The feedthrough 308 can have length 512 of about 0.175 to 0.185 inches, or ideally about 0.180 inches, or substantially the same as diameter 508 of the nailhead 404. Such a similarity in dimensions reduces the ease with which feedthrough 308 will bend or break during manufacturing, thereby reducing or eliminating the likelihood of electrical shorts or other failures by the feedthrough 308 contacting the top cover 306.

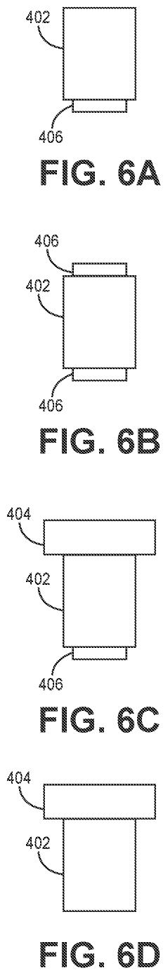

FIG. 6A is a side view of a pin 402 according to a first example embodiment. In the example illustrated in FIG. 6A, the pin 402 includes a shoulder feature 406 at a first end. FIG. 6B is a side view of a pin 402 according to a second example embodiment. In the example illustrated in FIG. 6B, the pin 402 includes a shoulder feature 406 at each of a first end and a second end. FIG. 6C is a side view of a pin 402 according to a third example embodiment. In the example illustrated in FIG. 6C, the pin 402 includes a shoulder feature 406 at a first end and a nailhead 404 at a second end. Other combinations are possible than those shown in FIGS. 6A-6C, and examples illustrated should not be understood as limiting the embodiments to any particular configuration. FIG. 6D is a side view of a pin 402 according to a fourth example embodiment. In the example illustrated in FIG. 6D, the pin 402 includes a nailhead 404. Other combinations are possible than those shown in FIGS. 6A-6D, and examples illustrated should not be understood as limiting the embodiments to any particular configuration.

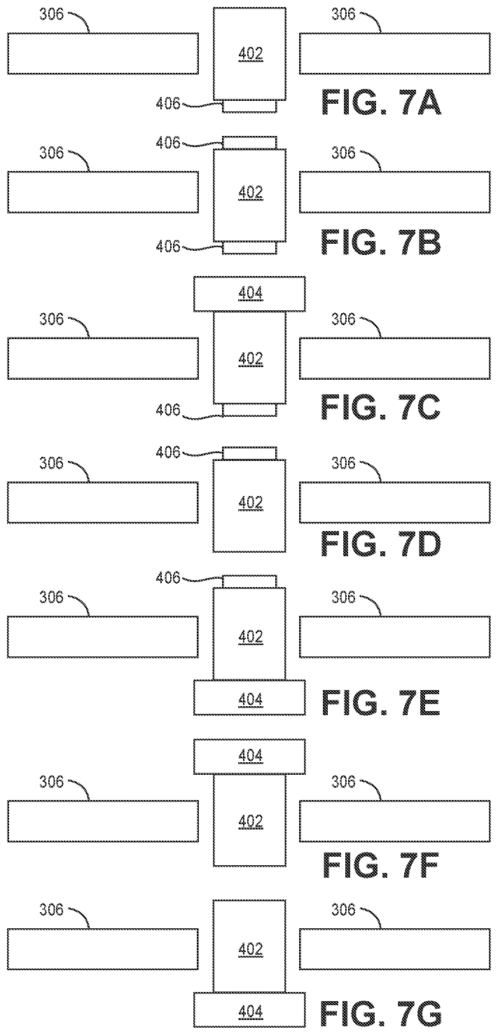

FIG. 7A illustrates a first assembly option in accordance with embodiments. In the example illustrated in FIG. 7A, a portion of pin 402 is above or outside the cover 306 and a portion including shoulder feature 406 is below or inside the cover 306. FIG. 7B illustrates a second assembly option in accordance with embodiments. In the example illustrated in FIG. 7B, a portion of pin 402 including a shoulder feature 406 is above or outside the cover 306 and a portion including a second shoulder feature 406 is below or inside the cover 306. FIG. 7C illustrates a third assembly option in accordance with embodiments. In the example illustrated in FIG. 7C, a portion of pin 402 including a nailhead 404 is above or outside the cover 306 and a portion including a shoulder feature 406 is below or inside the cover 306. FIG. 7D illustrates a fourth assembly option in accordance with embodiments. In the example illustrated in FIG. 7D, a portion of pin 402 including a shoulder feature 406 is above or outside the cover 306 and a portion of the pin 402 having neither a shoulder nor a nailhead is below or inside the cover 306. FIG. 7E illustrates a fifth assembly option in accordance with embodiments. In the example illustrated in FIG. 7E, a portion of pin 402 including a shoulder feature 406 is above or outside the cover 306 and a portion of the pin 402 having a nailhead 404 is below or inside the cover 306. FIG. 7F illustrates a sixth assembly option in accordance with embodiments. In the example illustrated in FIG. 7F, a portion of pin 402 is above or outside the cover 306 and a portion including a shoulder feature 406 is below or inside the cover 306. FIG. 7G illustrates a seventh assembly option in accordance with embodiments. In the example illustrated in FIG. 7G, a portion of pin 402 is above or outside the cover 306 and a portion including a shoulder feature 406 is below or inside the cover 306. Other combinations are possible than those shown in FIGS. 7A-7G, and examples illustrated should not be understood as limiting the embodiments to any particular configuration.

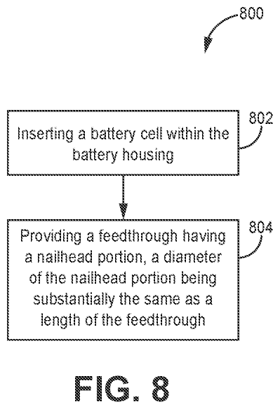

FIG. 8 is a flow diagram of a method 800 for manufacturing a battery 300 in accordance with embodiments. Reference is made to elements of the battery 300 described above with reference to FIGS. 3-5B. The method 800 can begin with operation 802 with inserting a battery cell 310 within a battery housing 302.

The method 800 can continue with operation 804 with providing a feedthrough 308 through an opening 324 in a top cover 306, the feedthrough 308 including a nailhead 404, the nailhead 404 having a diameter 508 substantially equal to a length 512 of the feedthrough 308. The feedthrough 308 can further include a pin 402 having a pin diameter 504 smaller than the diameter 508 of the nailhead 404. The feedthrough 308 can provided such that the pin 402 is substantially perpendicular to the top cover 306.

The method 800 can further comprise providing a shoulder feature 406 of the feedthrough 308 through a hole or other opening in a welding process. The shoulder feature 406 can have a shoulder diameter 500 smaller than the pin diameter 504. The method 800 can further include welding the top cover 306 to the tubular battery housing. The nailhead 404 can be used to retain an insulator or other material around the tubular battery housing and the nailhead 404 can provide a surface for further welding or coupling processes. A glass preform 408 can be provided and heated during the manufacturing process to provide a hermetical seal between the feedthrough 308 and the top cover 306.

Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Accordingly, the term “processor” as used herein may refer to any of the foregoing structure or any other physical structure suitable for implementation of the described techniques. Also, the techniques could be fully implemented in one or more circuits or logic elements.

What is claimed is:

1. A battery comprising:
a battery cell within a battery housing; and
a feedthrough inserted through an opening in a top cover of the battery, the feedthrough including, at least at one end of the feedthrough, at least one of a shoulder feature and a nailhead.

2. The battery of claim 1 wherein, if the battery includes the nailhead, the nailhead has substantially the same length as a length of the feedthrough.

3. The battery of claim 2, wherein the feedthrough further comprises a pin having a pin diameter smaller than the diameter of the nailhead.

4. The battery of claim 3, wherein if the battery includes the shoulder feature, the shoulder feature has a shoulder diameter smaller than the pin diameter.

5. The battery of claim 4, wherein a ratio of the pin diameter to a length of the pin is about 7.5:1.

6. The battery of claim 1, wherein the feedthrough is comprised of titanium.

7. The battery of claim 6, wherein the feedthrough is comprised of grade 23 titanium.

8. The battery of claim 1, wherein a diameter of the nailhead is about 0.175 inches.

9. The battery of claim 1, further comprising a glass preform between the top cover and the feedthrough.

10. A method for manufacturing a battery, the method comprising:
inserting a battery cell within a battery housing; and
providing a feedthrough through an opening in a top cover of the battery, the feedthrough including at least one of a nailhead and a shoulder feature.

11. The method of claim 10, wherein, if the battery includes the nailhead, a diameter of the nailhead is substantially the same as a length of the feedthrough.

12. The method of claim 10, wherein the feedthrough is provided such that a pin of the feedthrough is substantially perpendicular to the top cover.

13. The method of claim 12, wherein if the pin includes the shoulder feature, the method further comprises providing the shoulder feature to a jumper for a welding process.

14. The method of claim 13, wherein the shoulder feature has a shoulder diameter smaller than a pin diameter.

15. The method of claim 10, further comprising welding the top cover to the battery housing.

16. The method of claim 15, further comprising: using the nailhead to retain an insulator over the battery housing.

17. The method of claim 10, further comprising providing a glass preform between the feedthrough and the top cover.

18. The method of claim 17, further comprising heating the glass preform to provide a hermetical seal between the feedthrough and the top cover.

19. An implantable cardiac device (ICD) comprising:
a battery comprising:
a battery cell within a battery housing; and
a feedthrough inserted through an opening in a top cover of the battery, the feedthrough including a nailhead, a diameter of the nailhead being substantially the same as a length of the feedthrough.

20. The ICD of claim 19, wherein the feedthrough further comprises a pin having a pin diameter smaller than the diameter of the nailhead, and wherein the pin includes a shoulder feature, the shoulder feature having a shoulder diameter smaller than the pin diameter.
